(19) United States
(12) Patent Application Publication
(10) Pub. No.: US 2007/0073332 A1
(43) Pub. Date: Mar. 29, 2007

(54) Intraluminal filter having a cover sleeve

(75) Inventors: Sean Miller, Danvers, MA (US); Albert Dunfee, Byfield, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/235,968

(22) Filed: Sep. 26, 2005

(51) Int. Cl. A61M 29/00

(52) U.S. Cl. 606/200

(57) ABSTRACT

A temporary filtering device for collecting debris in a body lumen. An embolic filter is utilized that is expandable and collapsible by push-pull action. A filter proximal end is attached to a proximal shaft portion and a filter distal end is attached to a distal shaft portion. The proximal and distal shaft portions are slidably engaged within the filter, such that relative longitudinal movement between the proximal and distal shaft portions either moves the filter ends closer together to expand the filter, or moves the filter ends farther apart to collapse the filter. The filtering device includes a cover sleeve coaxially disposed about the filter that is attached to the distal shaft portion for aiding in the collapse of the filter. The cover sleeve may be attached to the distal shaft portion by one or more connecting rods, and is sized to slide over the filter and at least partially cover the filter openings, when the filter is being collapsed and when the filter is in its collapsed configuration. The temporary filtering device may be utilized without a distal shaft portion, wherein the filter distal end and connecting rods are attached or slidable relative to a core wire and relative movement between the core wire and proximal shaft reconfigures the filter between the expanded and collapsed configurations.

### FIELD OF THE INVENTION

The invention relates generally to intraluminal distal protection devices for capturing particulate in the vessels of a patient. More particularly, the invention relates to a filter for capturing emboli in a blood vessel during an interventional vascular procedure, the filter having a slidable cover sleeve for at least partially covering proximal openings of the filter in its collapsed configuration.

### BACKGROUND OF THE INVENTION

Catheters have long been used for the treatment of diseases of the cardiovascular system, such as treatment or removal of stenosis. For example, in a percutaneous transluminal coronary angioplasty (PTCA) procedure, a catheter is used to transport a balloon into a patient's cardiovascular system, position the balloon at a desired treatment location, inflate the balloon, and remove the balloon from the patient. Another example of a common catheter-based treatment is the placement of an intravascular stent in the body on a permanent or semi-permanent basis to support weakened or diseased vascular walls, or to avoid closure, re-closure or rupture thereof.

These non-surgical interventional procedures often avoid the necessity of major surgical operations. However, one common problem associated with these procedures is the potential release of embolic debris into the bloodstream that can occlude distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, pieces of plaque or clot material can sometimes dislodge from the stenosis during a balloon angioplasty procedure and become entrained in the bloodstream.

Medical devices have been developed to attempt to deal with the problem created when debris or fragments enter the circulatory system during vessel treatment. One technique includes the placement of a filter or trap downstream from the treatment site to capture embolic debris before it reaches the smaller blood vessels downstream. The placement of a filter in the patient's vasculature during treatment of the vascular lesion can collect embolic debris in the bloodstream.

It is known to attach an expandable filter to a distal end of a guidewire or guidewire-like member that allows the filtering device to be placed in the patient's vasculature. The guidewire allows the physician to steer the filter to a location downstream from the area of treatment. Once the guidewire is in proper position in the vasculature, the embolic filter can be deployed to capture embolic debris. Some embolic filtering devices utilize a restraining sheath to maintain the expandable filter in its collapsed configuration. Once the proximal end of the restraining sheath is retracted by the physician, the expandable filter will transform into its fully expanded configuration in apposition with the vessel wall. The restraining sheath can then be removed from the guidewire allowing the guidewire to be used by the physician to deliver interventional devices, such as a balloon angioplasty catheter or a stent delivery catheter, into the area of treatment. After the interventional procedure is completed, a recovery sheath can be delivered over the guidewire using over-the-wire techniques to collapse the expanded filter (with the trapped embolic debris) for removal from the patient's vasculature. Both the delivery sheath and recovery sheath should be relatively flexible to track over the guide wire and to avoid straightening the body vessel once in place.

Another distal protection device known in the art includes a filter mounted on a distal portion of a hollow guidewire or tube. A moveable core wire is used to open and close the filter. The filter is coupled at a proximal end to the tube and at a distal end to the core wire. Pulling on the core wire while pushing on the tube draws the ends of the filter toward each other, causing the filter framework between the ends to expand outward into contact with the vessel wall. Filter mesh material is mounted to the filter framework. To collapse the filter, the procedure is reversed, i.e., pulling the tube proximally while pushing the core wire distally to force the filter ends apart. A sheath catheter may be used as a retrieval catheter at the end of the interventional procedure to reduce the profile of the “push-pull” filter, as due to the embolic particles collected, the filter may still be in a somewhat expanded state. The retrieval catheter may be used to further collapse the filter and/or smooth the profile thereof, so that the filter guidewire may pass through the treatment area without disturbing any stents or otherwise interfering with the treated vessel.

However, regardless of how a distal protection filter is expanded during a procedure, i.e., sheath delivered or by use of a push-pull mechanism, a crossing profile of the collapsed filter is to be at a minimum to reduce interference between the filter and other interventional devices or in-placed stents. Further, once embolic debris is captured within the filter, there is a need in the art for a mechanism that prevents the debris from being pushed out of or released from the filter during filter collapse and recovery. Thus, what is needed is a filter that achieves a compact, reduced profile with effective embolic capture and retention.

### BRIEF SUMMARY OF THE INVENTION

The present invention is a filtering device having an embolic filter for collecting debris in a body lumen during an intravascular procedure. The filter is expandable and collapsible by push-pull action. A filter proximal end is attached to a proximal shaft portion and a filter distal end is attached to a distal shaft portion. The proximal and distal shaft portions are slidably engaged within the filter, and relative longitudinal movement there between either moves the filter ends closer together to expand the filter or moves the filter ends farther apart to collapse the filter. The filtering device includes a cover sleeve attached to the distal shaft portion and coaxially disposed about the filter. The cover sleeve may be attached to the distal shaft portion by one or more connecting rods. As the distal shaft portion is moved distally, it collapses the filter while it simultaneously draws the cover sleeve over the proximal portion of the filter. In this manner, the cover sleeve aids in the collapse of the filter from its proximal end. The cover sleeve also at least partially covers the filter openings during collapse and removal of the filtering device to reduce particulate discharge.

Another embodiment of the present invention is a filter guidewire for collecting debris in a body lumen that includes an elongate hollow guidewire, a core wire movably disposed within the hollow guidewire, and a filter mounted coaxially about the core wire. The filter has a proximal end disposed about and secured to the hollow guidewire, a distal end disposed and joined to the core wire and at least one opening for receiving debris near the filter proximal end, wherein relative longitudinal movement between the filter ends accompanies transformation of the filter between a collapsed configuration and a deployed configuration. The filter guidewire includes a cover sleeve coaxially disposed about the elongate hollow guidewire that includes at least one connecting rod with a distal portion extending within the filter and a distal end attached to the core wire. In another embodiment, the filter distal end and a distal end of the connecting rod are rotatably mounted to the core wire. In various embodiments of the present inventions, the cover sleeve may be fixedly or rotatably attached to the core wire by one or more connecting rods. As the core wire is moved distally relative to the proximal shaft, the filter is collapsed and the cover sleeve is drawn over the proximal portion of the filter to at least partially cover the filter opening(s). Whereas when the core wire is moved proximally relative to the proximal shaft, the filter is returned to its expanded configuration while the cover sleeve is slid proximally off of the filter opening(s).

In another embodiment of the present invention, the cover sleeve has a tapered inner diameter with the greatest diameter being at its distal end. In a further embodiment, the cover sleeve is connected to a distal end of the filter by one or more connecting rods that include a bowed portion positioned within the filter. In still a further embodiment of the present invention, the cover sleeve connecting rod is disposed through a guide opening in the filter.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a sectional side view of a filtering device in a deployed configuration in accordance with an embodiment of the present invention.

FIG. 2 is the filtering device of FIG. 1 in a collapsed configuration.

FIG. 3 is a sectional side view of a filtering device in a deployed configuration in accordance with another embodiment of the present invention.

FIG. 4 is the filtering device of FIG. 3 in a collapsed configuration.

FIG. 5 is a sectional side view of a filtering device in a deployed configuration in accordance with another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

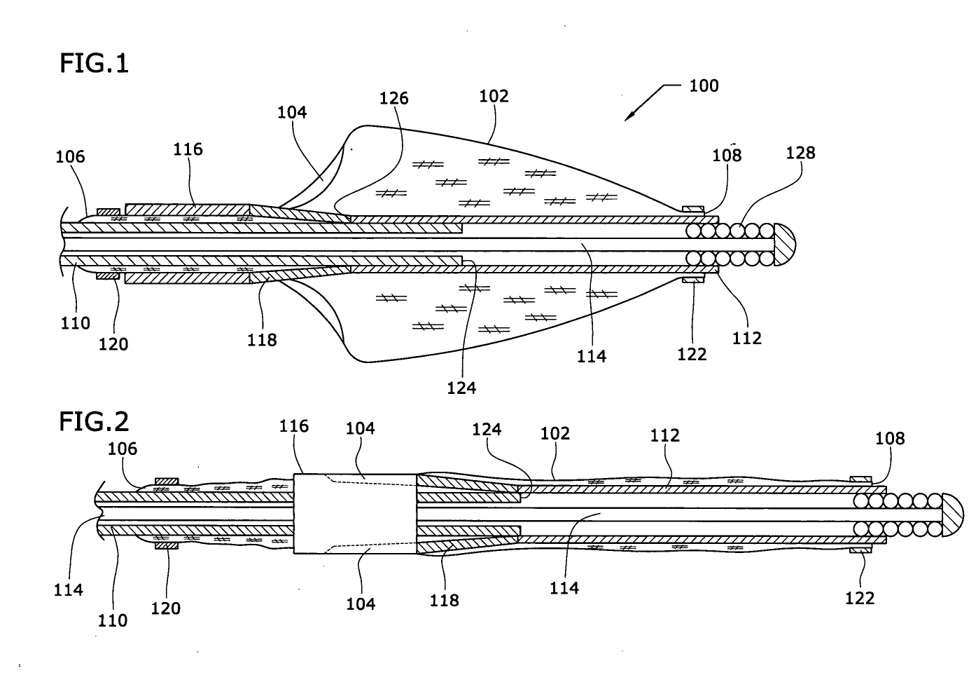

FIG. 1 illustrates a sectional side view of a portion of a filtering device 100. In accordance with this embodiment of the present invention, filtering device 100 is a temporary filter guidewire for use in intraluminal procedures in a manner similar to the filter guidewire devices disclosed in U.S. Pat. No. 6,866,677, which is incorporated by reference herein in its entirety. Filtering device 100 is shown in a deployed, i.e., expanded, configuration, such that the outer perimeter of filter 102 is in contact with an inner surface of a vessel wall when the device is in vivo. Filter 102 includes openings 104 in a proximal portion thereof for receiving embolic debris.

In an embodiment of the present invention, filter 102 is comprised of a plurality of wires or filaments that are woven together to form a tubular braided filter. The braiding wires or filaments are preferably made from stainless steel, a shape memory material such as nitinol, or a nickel-based super alloy. In another embodiment, filter 102 may be formed from a framework supporting a suitable mesh or porous material that collects embolic debris while permitting fluid to flow there through, such as blood flow sufficient for perfusion of body tissues. Such mesh filters and braided filters are disclosed in U.S. Pat. No. 6,346,116, which is incorporated by reference herein in its entirety

In the embodiment shown in FIG. 1, proximal end 106 is a cylindrical portion adjacent the proximal terminus of filter 102 and is fixedly attached along a distal portion of elongate proximal shaft 110. Distal end 108 is a cylindrical portion adjacent the distal terminus of filter 102 and is fixedly attached about a distal support shaft or tube 112. Distal support tube 112 may also be referred to as a transition sleeve because it serves to smooth the stiffness transition at distal end 124 of proximal shaft 110. Filter ends 106, 108 may be spot welded, laser welded or secured using a bonding sleeve or adhesive to proximal shaft 110 or distal support tube 112, respectively, as would be apparent to one skilled in the relevant art. In this embodiment, filter proximal end 106 and distal end 108 are surrounded by respective bands 120, 122, which may be sliding stops as described below and/or radiopaque markers to aid in fluoroscopic observation during manipulation of filtering device 100 through a patient's vasculature.

In another embodiment of the present invention, proximal shaft 110 may be constructed of multiple shaft components of varying flexibility to provide a gradual transition in flexibility as the shaft extends distally. Such a shaft arrangement is disclosed in U.S. Pat. No. 6,706,055, which is incorporated by reference herein in its entirety. In addition, a liner or axial bearings (not shown) as disclosed in the '055 patent may be utilized between core wire 114 and proximal shaft 110 or distal support tube 112 in order to facilitate sliding movement there between during expansion and collapse of filter 102. In another embodiment, proximal shaft 110 may be a hollow tube enabling filtering device 100 to also function as a medical guidewire.

Distal end 124 of proximal shaft 110 is slidably engaged within proximal end 126 of distal support tube 112. Coaxially disposed within proximal shaft 110 and support tube 112 is guidewire or core wire 114. Core wire 114 may be made from a shape memory metal, such as nitinol, or a stainless steel wire. In an embodiment of the present invention (not shown), core wire 114 may be tapered at its distal end and/or be comprised of one or more core wire sections. Core wire 114 has a proximal end (not shown) that extends outside of the patient from a proximal end of proximal shaft 110 (not shown), and a distal end that is fixed to tubular tip member 128. In the embodiment shown in FIG. 1, tip member 128 is a flexible coil spring made from thin wires of stainless steel and/or one of various radiopaque alloys such as platinum, as is well known to those of skill in the art of medical guidewires. Alternatively, tip member 128 may be comprised of a soft elastomeric material. Support tube 112 may extend distally from filter 102 and is fixedly or rotatably coupled at a distal end to tip member 128. Thus, longitudinal movement of filter distal end 108 is controlled by movement of core wire 114, which is transferred through intermediate components tip member 128 and support tube 112.

Filtering device 100 also includes cover sleeve 116 that is attached by two connecting rods 118 to the proximal portion of distal support tube 112. Distal ends of connecting rods 118 may be attached along the inside of, outside of or end-to-end with distal support tube 112 by spot welded, laser welded or secured using a bonding sleeve or adhesive, as would be apparent to one skilled in the relevant art. Cover sleeve 116 is a thin tubular member that is sized to slide or ride over filter 102 without adding significant bulk to and/or decreasing the flexibility of the underlying structure. Cover sleeve 116 is preferably as long as, or slightly shorter than, filter openings 104, and may be made from a thin biocompatible material, such as stainless steel or other metal, polyethylene, polyamide or other thermoplastic polymer, or polyimide or other thermoset polymer.

In the embodiment of FIG. 1, connecting rods 118 are disposed through respective filter openings 104, such that they are slidable therein. At each stage of operation of filter 102, cover sleeve 116 is positioned along the exterior of filter 102 with at least a portion of each connecting rod 118 being positioned within the filter. In various other embodiments of the present invention, cover sleeve 116 may be attached to distal support tube 112 by as many connecting rods 118 as there are filter openings or by as few as one connecting rod 118.

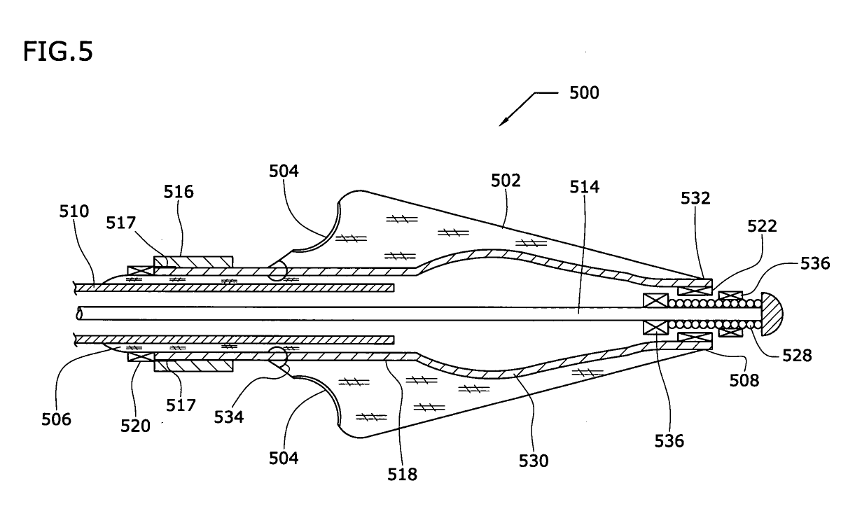

Connecting rods 118 are stressed under tension to draw cover sleeve 116 distally over filter 102, and are stressed under compression to slide cover sleeve 116 proximally off of filter openings 104. Such dual-purpose connecting rods 118 may be comprised of round, oval or flat cross-section stainless steel wire or another relatively stiff, biocompatible material. Instead of being disposed through filter openings 104, connecting rods 118 may slidably extend through dedicated guide openings in filter 102 that lie close to the longitudinal axis of the device, as shown in the embodiment of FIG. 5. Such guide openings can prevent rods 118 from bowing or flexing out of alignment, especially under compression loading, as the guide openings assist the connecting rods to remain relatively parallel to the longitudinal axis of the device at all times.

Connecting rods 118 may be attached directly to the distal end of cover sleeve 116 using a butt joint. Alternatively, connecting rods 118 may be cut from the same tubing that also defines cover sleeve 116, such that no joints are required between rods 118 and sleeve 116. In such an alternative embodiment, unitary sleeve 116 having connecting rods 118 extending there from may be made using miniature tubing fabrication techniques such as electric discharge (EDM) or laser machining, chemical etching, cutting, grinding, or slotting. The unitary sleeve 116 having connecting rods 118 may also be made by casting a thermoset polymer in the desired final shape. Such a unitary sleeve 116 having connecting rods 118 may also be stamped from a flat sheet of material and then rolled and joined at the edges to form cylindrical cover sleeve 116. In another embodiment, connecting rods 118 and distal support tube 112 may be made as a unitary component in a manner similar to that described for make the unitary component of cover sleeve 116 with connecting rods 118.

In FIG. 1, filter 102 is shown in the deployed configuration with cover sleeve 116 coaxially disposed about proximal end 106 of filter 102. FIG. 2 illustrates filtering device 100 in a collapsed configuration with cover sleeve 116 moved distally along filter 102 to a position substantially covering filter openings 104. Cover sleeve 116 is sized to slide over the proximal portion of filter 102 to aid in collapsing the filter and to reduce particulate discharge by at least partially covering filter openings 104 during filter collapse and removal. Filter 102 is collapsed by advancing core wire 114 within proximal shaft 110 to distally displace tip member 128 away from proximal shaft 110. The axial translation of tip member 128 draws support tube 112 distally along, but not out of engagement with, the distal portion of proximal shaft 110. As support tube 112 is drawn distally, connecting rods 118 slide through openings 104 pulling cover sleeve 116 distally over the exterior of filter 102. Accordingly, to transform filter 102 from its expanded configuration to its collapsed configuration, relative longitudinal movement of support tube 112 with respect to proximal shaft 110 causes filter distal end 108 to move away from filter proximal end 106 while drawing cover sleeve 116 distally over filter 102 and filter openings 104.

In an embodiment of the present invention wherein connecting rods 118 neither overlap nor underlap sleeve 116, rods 118 may prevent the distal end of cover sleeve 116 from sliding distally beyond filter openings 104 because cover sleeve 116 slides on the exterior of filter 102 while rods 118 are drawn within filter 102. Thus, as cover sleeve 116 approaches the distal edges of openings 104, filter 102 may ride up over rods 118, which act like ramps. An alternative embodiment that does not result in the “ramping-up” of the filter includes attachment of the proximal ends of connecting rods 118 inside cover sleeve 116 at a distance spaced from the distal end of cover sleeve 116 to form an underlapping joint. In this embodiment, the distalmost end of filter openings 104 may be drawn within cover sleeve 116 between an inside surface of the sleeve and the connecting rods, instead of being forced to ride outward over rods 118. A similar result can be accomplished with the unitary sleeve/rod construction described above by slitting cover sleeve 116 to extend the edges of connecting rods 118 proximally a distance into sleeve 116. In such an embodiment, rods 118 deflect inwardly along the slits to permit the distalmost end of filter openings 104 to be drawn within cover sleeve 116. In yet another embodiment, as shown in FIG. 5, connecting rods 118 can extend within cover sleeve 116 for attachment at or near the proximal end thereof.

Filtering device 100 traverses the vascular anatomy in its collapsed configuration. Cover sleeve 116 aids in maintaining filter 102 in its collapsed configuration while the filtering device is being tracked to and from the treatment site. Cover sleeve 116 also decreases the outer diameter of filter 102 by compressing filter 102 about proximal shaft 110 to a lower profile than customarily may be attainable by maintaining the filter in its collapsed configuration strictly by applying longitudinal tension on filter 102.

Filter 102 is expanded at the treatment site by withdrawing core wire 114 proximally through proximal shaft 110 to move filter ends 106, 108 closer together, causing transformation of filter 102 into the expanded configuration. This withdrawal of core wire 114 causes simultaneous proximal movement of support tube 112, tip member 128, connecting rods 118 and cover sleeve 116 relative to proximal shaft 110, such that openings 104 are uncovered and operationally positioned to receive embolic debris. The expansion of filter 102 stops when the filter is in apposition with the vessel wall of the patient. Surface contact is maintained around the entire vessel lumen to prevent any emboli from slipping past filter 102 during the procedure.

When an intravascular treatment is complete, embolic filter 102, which now contains embolic debris, must be collapsed and removed from the patient with preferably little to no release of captured particulate. In the present invention, filter 102 is mechanically collapsed by the push-pull mechanism previously discussed above. Accordingly, as filter 102 is drawn down by distal movement of core wire 114 and support tube 112 relative to proximal shaft 110, collapse of the proximal portion of filter 102 is concurrently assisted by cover sleeve 116 being slid distally there over, and filter openings 104 are at least partially covered by sleeve 116. By simultaneously covering the filter openings while closing/collapsing the filter, the possibility that particulate will be released is decreased. Thus, cover sleeve 116 functions not only to aid in the collapse of the filter, but also to reduce particulate discharge by covering the filter openings during the collapse. Filter openings 104 also remain covered by cover sleeves 116 while the filtering device is being pulled out of the patient's tortuous anatomy, thereby aiding the maintenance of the debris within filter 102 during removal.

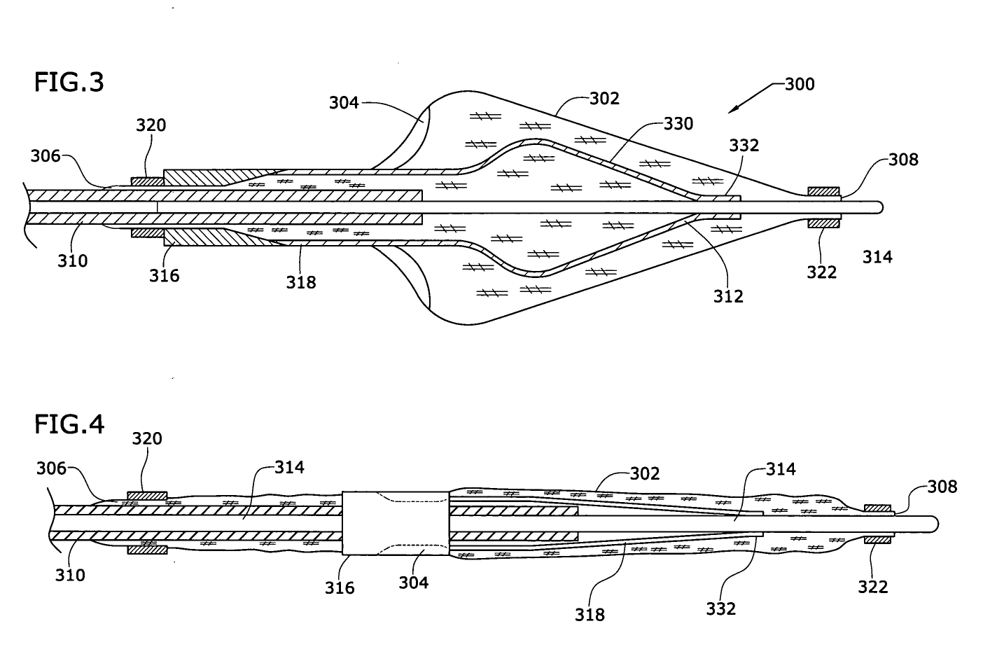

FIGS. 3 and 4 illustrate filtering device 300 according to another embodiment of the present invention. Filtering device 300 includes filter 302 having filter openings 304 in a proximal portion thereof. Proximal end 306 and distal end 308 of filter 302 are surrounded by bands 320 and 322, respectively, and are fixedly secured to proximal shaft 310 and core wire 314, respectively. Filter ends 306, 308 may be spot welded, laser welded or secured using a bonding sleeve or adhesive to proximal shaft 310 or core wire 314, respectively, as would be apparent to one skilled in the relevant art.

In the deployed configuration shown in FIG. 3, cover sleeve 316 is positioned over filter proximal end 306 with connecting rods 318 extending through filter openings 304. The inner diameter of cover sleeve 316 has a slight taper. The greater inner diameter being at the distal end of cover sleeve 316 aids in initiating proximal movement of the cover sleeve during filter expansion. In another embodiment, the tapered inner diameter of cover sleeve 316 may extend from a proximal end to a distal end thereof.

In comparison to filtering device 100, filtering device 300 also provides reduced axial travel of cover sleeve 316 during opening and closing of filter 302, which permits an advantageously shorter overall length of the filter device. Cover sleeve 316 slides axially over filter 302 without sliding off of filter proximal end 306. Thus, filter proximal end 306 must be long enough to accommodate the axial travel of cover sleeve 316. Filter proximal end 306 can be comparatively shorter than filter proximal end 106 because cover sleeve 316 travels a shorter axial distance than cover sleeve 116 for the reasons described below. In alternative embodiments of the invention, the cover sleeve may slide on and off the proximal end of the filter, including sliding over the proximal band.

In the embodiment shown in FIGS. 3 and 4, each of connecting rods 318 extends to a location near distal end 308 of filter 302 and includes a preformed bowed portion 330 positioned within the interior of the filter. Connecting rods 318 may be formed with a mechanical memory that can be imparted to the metal comprising rods 318 by thermal treatment to achieve a spring temper in stainless steel, for example, or to set a shape memory in a susceptible metal alloy, such as nitinol. Distal ends 332 of connecting rods 318 are fixedly joined to core wire 314 adjacent filter distal end 308. In another embodiment, connecting rod distal ends 332 may be attached to core wire 314 within the filter distal end joint. Filtering device 300 is expanded and collapsed in a similar manner as described above with reference to filtering device 100. However, during the collapse of filter 302, the axial translation of core wire 314 pulls connecting rods 318 to substantially straighten bowed portions 330, as shown in FIG. 4.

During expansion/opening of filter 302, bowed portions 330 resume their preformed bowed shapes. This bowing or lateral deflection of bowed portions 330 absorbs some of the axial length of connecting rods 318, thus reducing the distance that cover sleeve 316 is moved proximally during filter deployment. That is, cover sleeve 316 will translate a shorter axial distance than core wire 314 is translated. In comparison, cover sleeve 116 will translate substantially the same axial distance as support tube 112 because connecting rods 118 are maintained in a generally straight configuration during use of filtering device 100. Reducing the axial distance that cover sleeve 316 moves serves to shorten the overall length of filtering device 300, and/or prevents the cover sleeve from moving over or getting caught on proximal band 320 on filter proximal end 306.

Alternatively, as shown in filter guidewire 500 in FIG. 5, connecting rods 518 can be provided in a normally straight shape, similar to connecting rods 118. In this embodiment, proximal band 520 on filter proximal end 506 serves as a proximal stop to prevent farther proximal travel of cover sleeve 516 during filter deployment. After sleeve 516 contacts proximal band 520, further proximal movement of core wire 514 forces connecting rods 518 into the shape shown in bowed portions 530. In this embodiment, connecting rods 518 extend through guide openings 534 in filter 502. Guide openings 534 are disposed between filter openings 504 close to proximal shaft 510, such that connecting rods 518 are guided parallel to the central axis of the filtering device. Connecting rods 518 extend within cover sleeve 516 and are attached to the proximal end thereof at joints 517, such that rods 518 sliding through guide openings 534 will not interfere with cover sleeve 516 sliding completely over filter openings 504.

In the embodiment of FIG. 5, filter distal end 508 and connecting rod distal end 532 are rotatably attached to core wire 514. Cylindrical band 522 is attached within filter distal end 508 and is also attached to connecting rod distal end 532 such that core wire 514 may rotate relative to both filter 502 and connecting rods 518. Fixed stop 536 is attached to core wire 514 proximally of ring 522 to provide axial control of filter distal end 508 while allowing substantially free rotation of core wire 514 within filter 502. Flexible tip 528 is attached to core wire 514 distally of ring 522 to provide axial control of filter distal end 508 while allowing substantially free rotation of core wire 514 within filter 502. To collapse filter 502, core wire 514 is translated distally relative to proximal shaft 510, wherein proximal fixed stop 536 contacts ring 522, sliding it distally until filter 502 is in a collapsed configuration with cover sleeve 516 covering openings 504. To expand filter 502, core wire 514 is drawn proximally relative to proximal shaft 510, which allows flexible tip 528 to contact ring 522, sliding it proximally until filter 502 attains its expanded configuration. Concurrently with the expansion of filter 502, cover sleeve 516 moves proximally until it contacts stop 520, as discussed above.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A filtering device for collecting debris in a body lumen, comprising:
a proximal shaft having a distal end;
a distal support shaft having a proximal end slidably engaged with the distal end of the proximal shaft;
a filter having a proximal end secured about the proximal shaft, a distal end secured about the distal support shaft and an opening for receiving debris in a proximal portion of the filter; wherein relative longitudinal movement between the proximal shaft and the distal support shaft accompanies transformation of the filter between a collapsed configuration and a deployed configuration; and
a cover sleeve coaxially disposed about the proximal shaft and attached to the distal support shaft, wherein the cover sleeve aids in the collapse of the filter.

2. The filtering device of claim 1, wherein the cover sleeve at least partially covers the filter opening when the filter is in the collapsed configuration.

3. The filtering device of claim 1, wherein a connecting rod attaches the cover sleeve to the distal support shaft.

4. The filtering device of claim 3, wherein the connecting rod is disposed through the filter opening.

5. The filtering device of claim 3, wherein the connecting rod is attached to the distal support shaft adjacent the distal end of the filter.

6. The filtering device of claim 5, wherein the connecting rod includes a bowed portion positioned within the filter.

7. The filtering device of claim 1, wherein the cover sleeve has a tapered inner diameter.

8. The filtering device of claim 7, wherein the inner diameter is greatest at a distal end of the cover sleeve.

9. The filtering device of claim 1, wherein the cover sleeve is coaxially disposed about the proximal end of the filter when the filter is in the deployed configuration.

10. The filtering device of claim 9, further comprising: a proximal stop fixed about the filter proximal end for preventing proximal movement of the cover sleeve off of the filter proximal end during expansion of the filter.

11. The filtering device of claim 10, wherein the proximal stop is a radiopaque marker band.

12. A filter guidewire for collecting debris in a body lumen, comprising:
an elongate hollow guidewire;
a core wire movably disposed within the hollow guidewire and having a distal end extending there from;
a filter mounted coaxially about the core wire, the filter having a proximal end disposed about the hollow guidewire, a distal end disposed about the core wire and an opening for receiving debris near the filter proximal end, wherein relative longitudinal movement between the filter ends accompanies transformation of the filter between a collapsed configuration and a deployed configuration; and
a cover sleeve coaxially disposed about the elongate hollow guidewire having at least one connecting rod with a distal portion extending within the filter, wherein the cover sleeve aids in the collapse of the filter.

13. The filter guidewire of claim 12, wherein the cover sleeve at least partially covers the filter opening when the filter is in the collapsed configuration.

14. The filter guidewire of claim 12, wherein the distal end of the filter is joined to the core wire.

15. The filter guidewire of claim 14, wherein the proximal end of the filter is secured to the hollow guidewire and wherein relative longitudinal movement between the hollow guidewire and the core wire transforms the filter between the collapsed and deployed configurations.

16. The filter guidewire of claim 14, wherein a distal end of the connecting rod is attached to the core wire proximal to the distal filter joint.

17. The filter guidewire of claim 16, wherein the connecting rod is disposed through a guide opening in the filter.

18. The filter guidewire of claim 14, wherein a distal end of the connecting rod is attached to the core wire within the distal filter joint.

19. The filter guidewire of claim 18, wherein the connecting rod includes a bowed portion positioned within the filter.

20. The filter guidewire of claim 12, wherein the distal end of the filter and a distal end of the connecting rod are rotatably mounted to the core wire.

21. The filter guidewire of claim 20, wherein the proximal end of the filter is secured to the hollow guidewire and wherein relative longitudinal movement between the hollow guidewire and the core wire transforms the filter between the collapsed and deployed configurations.
